(19) United States
(12) Patent Application Publication
(10) Pub. No.: US 2025/0166752 A1
(43) Pub. Date: May 22, 2025

(54) METHOD, PROGRAM, AND DEVICE FOR PROVIDING ARTIFICIAL INTELLIGENCE-BASED MULTILINGUAL MEDICAL HISTORY SUMMARIZATION AND TRANSLATION SERVICE

(71) Applicant: Mediark Inc., Seoul (KR)

(72) Inventors: Chan Hyung Lee, Seoul (KR); Ban Hyung Lee, Seoul (KR); Hyun Sung Lee, Gyeonggi-do (KR); Jung Do Park, Seoul (KR)

(21) Appl. No.: 18/767,901

(22) Filed: Jul. 9, 2024

(30) Foreign Application Priority Data
Nov. 20, 2023 (KR) 10-2023-0160350

(51) Int. Cl. G16H 10/60; G06F 40/166; G06F 40/289; G06F 40/58; G16H 15/00; G16H 50/70

(52) U.S. Cl. CPC G16H 10/60; G06F 40/58; G16H 15/00; G16H 50/70

(57) ABSTRACT

An artificial intelligence (AI)-based multilingual medical examination summarization method is performed by a processor of a server, and includes generating medical examination data to which unique IDs have been assigned, respectively, wherein the medical examination data comprise user's personal information and symptom and/or disease-related data of the user, obtaining language selection information that is information of a first language, selected among preset languages, from a user terminal, obtaining answer data for the medical examination data corresponding to the language selection information from the user terminal, and classifying all of the answer data based on a unique ID corresponding to a preset item and generating summarization data by extracting a linguistic expression in the forms of the first language and terms corresponding to the preset item.

### BACKGROUND

The present disclosure relates to a multilingual medical examination summarization and translation method, program, and apparatus, and more particularly, to a method, program, and apparatus that provide a service in which a medical examination and an answer to a medical examination are summarized in multiple languages and are translated into other languages based on artificial intelligence.

As people's medical-related recognition is increased, the importance of the summarization and organization of medical examination-related data after medical examinations is increased in the medical field. However, a medical examination record organization and summarization system for sharing information with medical teams and patients are not sufficient.

As the medical field is a professional field, it is not easy to clearly describe even a medical team that is expressed in a mother language in addition to a foreign language except a person who has learned special knowledge. Furthermore, if a sentence that is expressed in a mother language is translated through the existing translation system, there is a good possibility that the sentence will not be translated according to a user's intention in view of the characteristics of medical teams. In addition, although a patient has symptoms in a foreign country, it is difficult for the patient to take medical treatment after visiting a hospital in the foreign country due to a language barrier. Although the patient takes medical treatment, misdiagnosis may be made due to misunderstanding between the patient and a medical team.

Accordingly, there is a need for a method of performing the summarization of medical examinations and translating an abridgement for each language anywhere at any time, in which when symptoms occur, medical examinations can be performed on a patient in multiple languages and the patient takes medical treatment without a language barrier even in a foreign country.

### SUMMARY

Various embodiments are directed to providing a method, program, and apparatus that provide a service in which medical examination data are classified based on unique IDs by assigning the unique IDs to the medical examination data, respectively, a multilingual version of summarization data are generated by extracting the medical examination data in forms of terms for each language, and the summarization data are translated.

Technical objects to be achieved by the present disclosure are not limited to the aforementioned object, and the other objects not described above may be evidently understood from the following description by a person having ordinary knowledge in the art to which the present disclosure pertains.

In an embodiment, an artificial intelligence (AI)-based multilingual medical examination summarization method is performed by a processor of a server, and may include generating medical examination data to which unique IDs have been assigned, respectively, wherein the medical examination data comprise user's personal information and symptom and/or disease-related data of the user, obtaining language selection information that is information of a first language, selected among preset languages, from a user terminal, obtaining answer data for the medical examination data corresponding to the language selection information from the user terminal, and classifying all of the answer data based on a unique ID corresponding to a preset item and generating summarization data by extracting a linguistic expression in the forms of the first language and terms corresponding to the preset item.

In an embodiment, the unique ID may include a first classification code to primarily classify the medical examination data into a plurality of types, and a second classification code to secondarily classify the primarily classified medical examination data into a plurality of types corresponding to each of the first classification codes.

In an embodiment, the generating of the summarization data may include classifying all of the answer data based on the first classification code corresponding to the preset item, classifying and arranging the answer data that have been classified for each preset item based on the second classification code, extracting the classified answer data in the forms of (the first language corresponding to the language selection information and the terms corresponding to the preset item, and generating a sentence in the first language based on the language selection information by connecting the extracted terms.

In an embodiment, the extracting of the classified answer data in the first language corresponding to the language selection information and the form of the term corresponding to the preset item may include extracting the answer data in the first language corresponding to the language selection information and the form of the term corresponding to the preset item, among a plurality of pre-stored linguistic expressions corresponding to the unique ID of the answer data.

In an embodiment, the generating of the sentence in the first language based on the language selection information by connecting the extracted terms may include AI training. The AI training may include performing AI training by using all terms extracted for each preset item as input data and using a sentence completed in the first language as output data.

In an embodiment, the first classification code may be a code to classify the medical examination data as one of an independent data type that is a type in which an additional description is required, a dependent data type that is a type in which the independent data type is described, a personal data type for the user's personal information, and other data type that is a type that does not require an additional description and in which the independent data type is not described.

In an embodiment, the second classification code may be a code to classify the primarily classified medical examination data into a data type for a plurality of pieces of aspect information related to the types classified by the first classification code, respectively.

In an embodiment, the method may further include translating the summarization data. The translating of the summarization data may include obtaining, from the user terminal, translation request information that is a request to translate the summarization data from the first language to a second language except the first language, among the preset languages, extracting the answer data in a form of terms of the second language corresponding to the unique ID of the answer data based on the translation request information, and generating a sentence in the second language by connecting the extracted terms in the second language.

In an embodiment, the obtaining of the answer data may include distinguishing between first answer information that is selected by the user and second answer information that is not selected, within the medical examination data, and obtaining, from the user terminal, answer data including the first answer information and/or the second answer information for the medical examination data.

In an embodiment, the obtaining of the answer data may include obtaining information on a first symptom of the user from the user terminal, obtaining, from the user terminal, information on a second symptom of the user that is a symptom accompanying the first symptom, classifying question information regarding the user, among pieces of pre-stored question information based on the information obtained from the user terminal, transmitting the classified question information to the user terminal, and obtaining answer information for the question information from the user terminal.

In an embodiment, there is provided a computer program stored in a computer-readable storage medium, wherein when the computer program is executed by a processor of an apparatus, an artificial intelligence (AI)-based multilingual medical examination summarization method may be performed by a processor of an apparatus and may include generating medical examination data to which unique IDs have been assigned, respectively, wherein the medical examination data comprise user's personal information and symptom and/or disease-related data of the user, obtaining language selection information that is information of a first language, selected among preset languages, from a user terminal, obtaining, from the user terminal, answer data for medical examination data corresponding to the language selection information, and classifying all of the answer data based on a unique ID corresponding to a preset item and generating summarization data by extracting a linguistic expression in a first language and forms of term corresponding to the preset item.

In an embodiment, the unique ID may include a first classification code to primarily classify the medical examination data into a plurality of types, and a second classification code to secondarily classify the primarily classified medical examination data into a plurality of types corresponding to each of the first classification codes.

In an embodiment, the generating of the summarization data may include classifying all of the answer data based on the first classification code corresponding to the preset item, classifying and arranging the answer data that have been classified for each preset item based on the second classification code, extracting the classified answer data in the forms of (the first language corresponding to the language selection information and the terms corresponding to the preset item, and generating a sentence in the first language based on the language selection information by connecting the extracted terms.

In an embodiment, an artificial intelligence (AI)-based multilingual medical examination summarization apparatus may include a storage unit in which at least one program instruction is stored and a processor configured to perform the at least one program instruction. The processor may generate medical examination data to which unique IDs have been assigned, respectively, wherein the medical examination data comprise user's personal information and symptom and/or disease-related data of the user, may obtain language selection information that is information of a first language, selected among preset languages, from a user terminal, may obtain answer data for the medical examination data corresponding to the language selection information from the user terminal, and may classify all of the answer data based on a unique ID corresponding to a preset item and may generate summarization data by extracting a linguistic expression in the forms of the first language and terms corresponding to the preset item.

In an embodiment, the unique ID may include a first classification code to primarily classify the medical examination data into a plurality of types and a second classification code to secondarily classify the primarily classified medical examination data into a plurality of types corresponding to each of the first classification codes.

In an embodiment, in generating the summarization data, the processor may classify all of the answer data based on the first classification code corresponding to the preset item, may classify and arranges the answer data that have been classified for each preset item based on the second classification code, may extract the classified answer data in the forms of (the first language corresponding to the language selection information and the terms corresponding to the preset item, and may generate a sentence in the first language based on the language selection information by connecting the extracted terms.

The aspects of the present disclosure are merely some of preferred embodiments of the present disclosure, and a person having ordinary knowledge in the field may derive and understand various embodiments into which technical characteristics of the present disclosure have been incorporated from the detailed description of the present disclosure to be described hereinafter.

According to an embodiment of the present disclosure, the server generates summarization data by classifying and arranging answer data for each of items within the summarization data by using unique IDs. Accordingly, efficiency and productivity of medical treatment can be improved because the time that is taken for medical examinations and recording and writing, which occupy most of the medical treatment time, is reduced. Furthermore, medical records can be standardized because the server has a preset structure for each of items within summarization data based on unique IDs, and the amount of records can be increased due to efficiency of medical records.

Furthermore, a multilingual version of summarization data can be derived because the server generates summarization data by extracting a linguistic expression corresponding to each item within the summarization data by using a unique ID through the system according to an embodiment of the present disclosure. Accordingly, a user (or patient) can easily receive a medical service although the user (or patient) is in a place where there is a language barrier because verbal communication between the user (or patient) and a medical team is easily performed. Furthermore, misdiagnosis by a medical team and the misuse and overuse of medical drugs can be minimized because information that is missed due to a language barrier between a user and the medical team can be minimized.

However, effects of the present disclosure which may be obtained in the present disclosure are not limited to the aforementioned effects, and other effects not described above may be evidently understood by those skilled in the art from the following description.

### BRIEF DESCRIPTION OF THE DRAWINGS

FIG. 1 is a conceptual diagram illustrating an embodiment of a construction of a system that provides an artificial intelligence (AI)-based multilingual medical examination summarization and translation service.

FIG. 2 is a block diagram illustrating an embodiment of a construction of a device that constitutes the system for providing the AI-based multilingual medical examination summarization and translation service.

FIG. 3 is a conceptual diagram illustrating an embodiment of an artificial neural network that is included in the device of the system that provides the AI-based multilingual medical examination summarization and translation service.

FIG. 4 is a diagram illustrating a structure of the system that provides the AI-based multilingual medical examination summarization and translation service.

FIG. 5 is a flowchart illustrating an embodiment of a method of providing the AI-based multilingual medical examination summarization and translation service.

FIG. 6 is a flowchart illustrating an embodiment of a method of providing a translation service by the system that provides the AI-based multilingual medical examination summarization and translation service.

FIG. 7 is a flowchart illustrating an embodiment of a method of providing diagnosis information by the system that provides the AI-based multilingual medical examination summarization and translation service.

FIG. 8 is a diagram illustrating an embodiment of interfaces for obtaining information on a first symptom of a user, which is output to a user terminal.

FIG. 9 is a diagram illustrating an embodiment of an interface for obtaining information on a second symptom.

FIG. 10 is a diagram illustrating an embodiment of interfaces for displaying question information regarding a symptom and/or disease of a user, which is output to the user terminal.

FIG. 11 is a diagram illustrating an embodiment of interfaces for displaying question information regarding a symptom and/or disease of a user, which is output to the user terminal.

FIG. 12 is a diagram illustrating an embodiment of interfaces for obtaining information on the symptom and/or disease-related history of a user, which is output to the user terminal.

FIG. 13 is a diagram illustrating an embodiment of interfaces for obtaining symptom and/or disease-related additional information of a user, which is output to the user terminal.

FIG. 14 is a diagram illustrating an embodiment of interfaces for displaying summarization information and diagnosis information of a user, which are output to the user terminal.

FIG. 15 is a diagram illustrating an embodiment of an interface in which summarization information past histories of a user are expressed in Korean in order to output the summarization information past history of a user, which is output to the user terminal.

FIG. 16 is a diagram illustrating an embodiment of interfaces for obtaining language selection information of a user, which are output to the user terminal.

FIG. 17 is a diagram illustrating an embodiment of interfaces in which different languages are expressed in order to obtain information on a first symptom of a user, which is output to the user terminal.

FIG. 18 is a diagram illustrating an embodiment of an interface for outputting summarization information and diagnosis information of a user to the user terminal in English.

### DETAILED DESCRIPTION

The present disclosure may be changed in various ways and may have various embodiments. Specific embodiments are to be illustrated in the drawings and specifically described. It should be understood that the present disclosure is not intended to be limited to the specific embodiments, but includes all of changes, equivalents and/or substitutions included in the spirit and technical range of the present disclosure.

Terms, such as “first” and “second”, may be used to describe various components, but the components should not be restricted by the terms. The terms are used to only distinguish one component from another component. For example, a first component may be referred to as a second component without departing from the scope of rights of the present disclosure. Likewise, a second component may be referred to as a first component. The term “and/or” includes a combination of a plurality of related and described items or any one of a plurality of related and described items.

An expression of the singular number includes an expression of the plural number unless clearly defined otherwise in the context. That is, if the singular number is not specified otherwise or is not clearly indicated in the context, in the present disclosure and the claims, in general, the singular number should be interpreted as meaning “one or more”.

When it is described that one component is “connected” or “coupled” to the other component, it should be understood that one component may be directly connected or coupled to the other component, but a third component may exist between the two components. In contrast, when it is described that one component is “directly connected” or “directly coupled” to the other component, it should be understood that a third component does not exist between the two components.

Terms used in this application are used to merely describe a specific embodiment, and are not intended to limit the present disclosure. An expression of the singular number includes an expression of the plural number unless clearly defined otherwise in the context. In this application, it is to be understood that a term, such as “include” or “have”, is intended to designate that a characteristic, a number, a step, an operation, a component, a part or a combination of them described in the specification is present, and does not exclude the presence or addition possibility of one or more other characteristics, numbers, steps, operations, components, parts, or combinations of them in advance.

Terms “information” and “data” that are used in the present disclosure may be interchangeably used. Terms “linguistic expression” and “term” that are used in the present disclosure may be interchangeably used.

All terms used herein, including technical terms or scientific terms, have the same meanings as those commonly understood by a person having ordinary knowledge in the art to which the present disclosure pertains, unless defined otherwise in this application. Terms, such as those defined in commonly used dictionaries, should be construed as having the same meanings as those in the context of a related technology, and are not construed as ideal or excessively formal meanings unless explicitly defined otherwise in this application.

Hereinafter, preferred embodiments of the present disclosure are described in detail with reference to the accompanying drawings. In describing the present disclosure, in order to facilitate general understanding of the present disclosure, the same reference numeral is used for the same component, and a redundant description of the same component is omitted.

FIG. 1 is a conceptual diagram illustrating an embodiment of a construction of a system that provides an artificial intelligence (AI)-based multilingual medical examination summarization and translation service.

Referring to FIG. 1, the system that provides the AI-based multilingual medical examination summarization and translation service may include a server 110 that provides the AI-based multilingual medical examination summarization and translation service and a user terminal 130 that is connected to the server 110 over a network 120. The system that provides the AI-based multilingual medical examination summarization and translation service may be a system for collecting symptom information and/or disease information of a user over a wired or wireless communication network and summarizing and organizing the symptom information and/or disease information in multiple languages. Furthermore, the server 110 may be a system that provides a service that analyzes symptom information and/or disease information of a user and provides diagnosis information of a user, in addition to the AI-based multilingual medical examination summarization and translation service.

The server 110 may store information that is necessary to provide the AI-based multilingual medical examination summarization and translation service. The server 110 may previously store information on symptoms and diseases, medical examination data that are question information for symptoms and diseases, and information including an algorithm for summarizing and organizing answer data for medical examination data in multiple languages. In this case, the answer data are data including medical examination data and answer information for medical examination data of a user. Furthermore, the server 110 may store the existing summarization information of a user.

In this case, a unique ID is assigned to a question included in the medical examination data and an answer sheet for the question. The server 110 may generate summarization data by classifying and arranging answer data for medical examination data based on unique ID. The same unique ID as that of medical examination data is assigned to a question included in answer data and an answer sheet for the question because the answer data include the medical examination data.

The network 120 may mean a connection structure for exchanging pieces of information between the server 110 and the user terminal 130. The network 120 may include the Internet, a local area network (LAN), a wireless local area network (wireless LAN), a wide area network (WAN), a personal area network (PAN), 3G, 4G, long term evolution (LTE), voice over LTE (VoLTE), 5G new radio (NR) wireless-fidelity (Wi-Fi), Bluetooth, NFC, radio frequency identification (RFID), a home network, and Internet of things (IoT).

The user terminal 130 may be connected to the server 110 over the network 120. Furthermore, the user terminal 130 may include user equipment, such as a computer, a tablet, or a smartphone. The user terminal 130 may receive, from the server 110, information that is necessary to provide a service for summarizing medical examination results of a user. The user terminal 130 may receive medical examination data from the server 110, and may transmit answer data for medical examination data to the server 110. The server 110 may transmit summarization data to the user terminal 130 by summarizing and organizing answer data. Furthermore, the user terminal 130 may display medical examination data and answer data through an output device in a linguistic expression.

The server 110 and the user terminal 130 in FIG. 1 may be denoted as a device. The construction of the device may be described as follows.

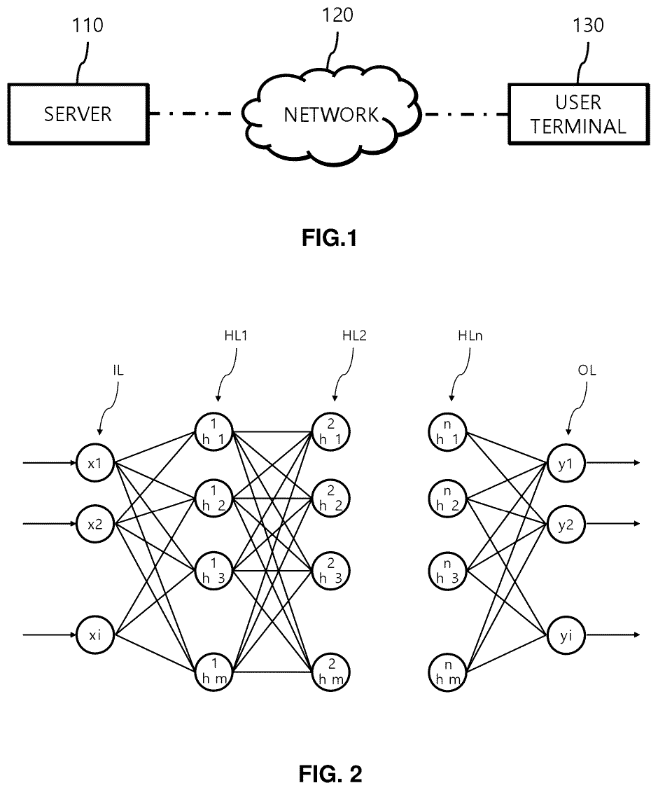

FIG. 2 is a block diagram illustrating an embodiment of a construction of a device that constitutes the system for providing the AI-based multilingual medical examination summarization and translation service.

A device 200 that is included in the system that provides the AI-based multilingual medical examination summarization and translation service may include at least one processor 210 and memory 220 that stores instructions to instruct the at least one processor 210 to perform at least one step.

In this case, the at least one processor 210 may mean a central processing unit (CPU), a graphics processing unit (GPU), or a dedicated processor that performs methods according to embodiments of the present disclosure. Each of the memory 220 and a storage device 260 may consist of at least one of a volatile storage medium and a nonvolatile storage medium. For example, the memory 220 may consist of at least one of read only memory (ROM) and random access memory (RAM).

Furthermore, the device 200 that is included in the system that provides the AI-based multilingual medical examination summarization and translation service may include a transceiver 230 that performs communication over a wireless network. Furthermore, the device 200 that is included in the system that provides the AI-based multilingual medical examination summarization and translation service may further include an input interface device 240, an output interface device 250, and the storage device 260. The components included in the device 200 that is included in the system that provides the AI-based multilingual medical examination summarization and translation service may be connected by a bus 270, and may communicate with each other.

Furthermore, the device 200 that is included in the system that provides the AI-based multilingual medical examination summarization and translation service may further include hardware components or software components for implementing/driving an artificial neural network. For example, the hardware components may include a neural processer unit (NPU). For example, the software components may include a framework, a kernel or a device driver, middleware, an application programming interface (API), and an application program (or application). An artificial neural network that is implemented in the device is described as follows.

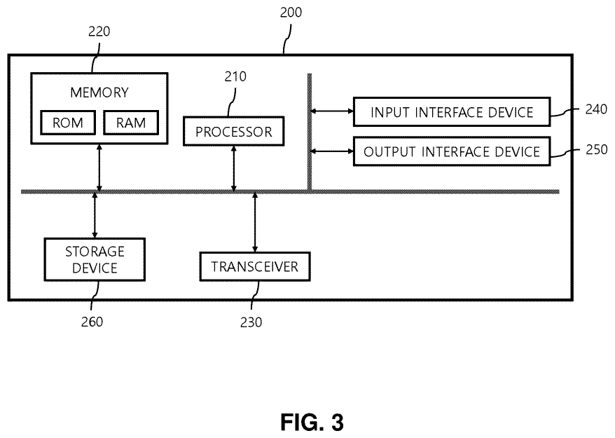

FIG. 3 is a conceptual diagram illustrating an embodiment of the artificial neural network that is included in the device of the system that provides the AI-based multilingual medical examination summarization and translation service.

Referring to FIG. 3, the artificial neural network may include an input layer IL, a plurality of hidden layers HL1, HL2 to HLn, and an output layer OL.

The input layer IL may include i (i is a natural number) input nodes x1, x2 to xi. Furthermore, vector input data having a length of i may be input to an input node.

The plurality of hidden layers HL1, HL2 to HLn includes n (n is a natural number) hidden layers, and may include hidden nodes h11, h12, h13 to h1m, h21, h22, h23, . . . , h2m, hn1, hn2, hn3, . . . , hnm. For example, the hidden layer HL1 may include m (m is a natural number) hidden nodes h11, h12, h13 to h1m. The hidden layer HL2 may include m hidden nodes h21, h22, h23 to h2m. The hidden layer HLn may include m hidden nodes hn1, hn2, hn3 to hnm.

The output layer OL may include j (j is a natural number) output nodes y1, y2 to yj corresponding to a class to be classified, and may output results (e.g., a score or a class score) for each class with respect to input data. The output layer OL may be called a fully connected layer.

The artificial neural network illustrated in FIG. 3 may include a branch between nodes that are shown in a straight line between two nodes, and weight values between connected nodes. In this case, nodes within one layer may not be connected, and nodes included in different layers may be fully or partially connected.

Each node (e.g., h11) in FIG. 3 may perform an operation by receiving the output of a previous node (e.g., x1), and may transmit the results of the operation to a subsequent node (e.g., h21). In this case, each of the nodes may calculate a value to be output by applying an input value to a specific function (e.g., a non-linear function).

In general, the structure of an artificial neural network is predetermined. Proper values of weights according to a branch between nodes may be calculated based on data correct answers of which have already been known regarding which class the weights belong to. Data correct answers of which have already been known may be denoted as “learning data”. A process of determining a weight may be denoted as “learning”. Furthermore, a bundle of structures and weights which may be independently trained may be denoted as a “model”.

That is, the device illustrated in FIG. 2, such as a server, may perform operations that are supported by the system by using the artificial neural network illustrated in FIG. 3. The structure of the system that provides the AI-based multilingual medical examination summarization and translation service may be described as follows.

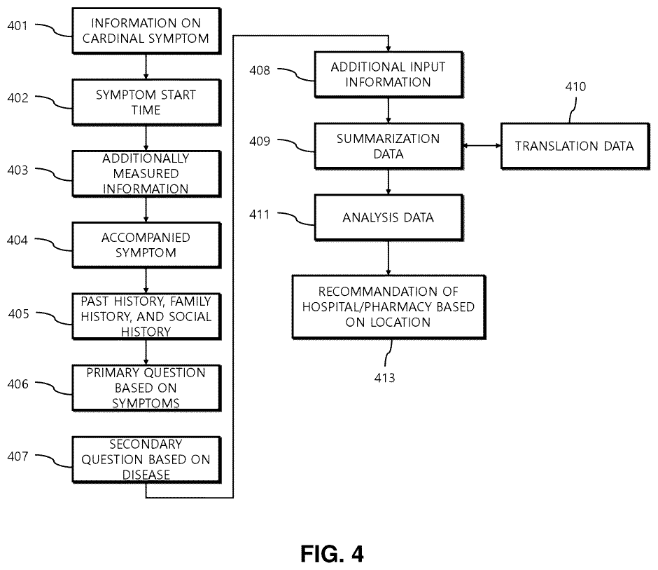

FIG. 4 is a diagram illustrating a structure of the system that provides the AI-based multilingual medical examination summarization and translation service.

Referring to FIG. 4, the system that provides the AI-based multilingual medical examination summarization and translation service may obtain symptoms of a user and/or disease-related information of a user. In this case, the symptoms of the user and/or the disease-related information of the user may include information 401 on a cardinal symptom of a user, the symptom start time 402 of a cardinal symptom, additional measuring information 403 of a user, information 404 on an accompanied symptom that accompanies a cardinal symptom of a user, and information 405 on the past history, family history, and social history of a user.

In this case, the additional measuring information is information of a vitals sign, and may include body temperature information, blood pressure information, pulse information, and respiration rate information of a user. Furthermore, the information on the past history of a user includes information on the disease history, surgical history, and procedural history of a user. The information on the social history of a user includes information on the occupation, smoking history, and drinking history of a user.

The system that provides the AI-based multilingual medical examination summarization and translation service may obtain information that is required for the medical examinations and diagnosis of a user by using a primary question 406 based on symptoms and a secondary question 407 based on a disease. In this case, the system that provides the AI-based multilingual medical examination summarization and translation service may obtain information on the primary question 406 based on symptoms and the secondary question 407 related to a disease, from a database that previously stores information on symptoms, information related to a disease, and information on a question about a symptom and/or a disease. Furthermore, the system that provides the AI-based multilingual medical examination summarization and translation service may obtain additional input information 408 of a user. In this case, the additional input information 408 may be descriptive information that is additionally input by a symptom and/or disease-related user in addition to questions for medical examination data.

In this case, information on symptoms, information on a disease, information on the primary question 406 based on symptoms, information on the secondary question 407 based on a disease, and information on multilingual version linguistic expressions related to symptoms and/or a disease may be recorded on the database.

The information on symptoms, which is recorded on the database, may include information on the name of a symptom, the emergency of a symptom, at least one accompanied symptom related to a symptom, at least one linguistic expression corresponding to a symptom, question information related to a symptom and/or a unique ID that is assigned to each of question data including question information. In this case, the at least one linguistic expression corresponding to the symptom may include an idiomatic expression for expressing a symptom, a plurality of forms of terms for expressing a symptom, and a term that expresses a symptom for each of a plurality of languages. In this case, the form of the term means that the term is expressed in a word form, an idiom form, or a sentence form in order to express a symptom. Accordingly, a unique ID assigned to each of question data may correspond to a plurality of idiomatic expressions, a plurality of forms of terms, and a term for each of a plurality of languages for one symptom.

The information on the disease that is recorded on the database may include information on the name of a disease, medical treatment associated with a disease, symptoms of a disease, at least one condition for determining a disease, question information related to a disease and/or a unique ID assigned to each of question data including question information. In this case, the at least one linguistic expression corresponding to the disease may include an idiomatic expression for expressing a disease, a plurality of forms of terms for expressing a disease, and a term that expresses a disease for each of a plurality of languages. Accordingly, the unique ID assigned to each of the question data may correspond to a plurality of idiomatic expressions, a plurality of forms of terms, and a term for each of a plurality of languages for one disease. In this case, the at least one condition for determining the disease may include an inclusive condition, that is, a condition regarding symptom that develop when a disease develops, and/or an exclusion condition, that is, a condition regarding symptoms that do not develop when a disease develops.

The information on the primary question 406 based on symptoms, which is recorded on the database, may include information, such as unique ID information for indicating a primary question based on symptoms, a question that is output, at least one answer sheet for a question, and answer information corresponding to an answer sheet. Furthermore, the information on the primary question 406 based on symptoms may further include information on an additional question that is associated with at least one answer sheet for the primary question 406 and/or information for indicating an additional question.

The information on the secondary question 407 based a disease, which is recorded on the database, may include information, such as unique ID information for indicating a secondary question based on a disease, a question that is output, at least one answer sheet for a question, and answer information corresponding to an answer sheet. Furthermore, the information on the secondary question 407 based on a disease may further include information on an additional question associated with an at least one answer sheet for the secondary question 407 and/or information for indicating an additional question.

The information on the multilingual version linguistic expressions related to symptoms and/or a disease, which is recorded on the database, may include a linguistic expression for each of a plurality of languages, which corresponds to one symptom and/or one disease.

The system that provides the AI-based multilingual medical examination summarization and translation service may obtain answer information, including the cardinal symptom-related information 401, the symptom start time 402, the additional measuring information 403, and the accompanied symptom-related information 404, from a user, may provide the user with information on a primary question based on symptoms and a secondary question based on a disease, and may obtain answer information for the questions. That is, the system that provides the AI-based multilingual medical examination summarization and translation service may provide medical examination data to the user and obtain answer data for the medical examination data from the user.

In an embodiment, the system that provides the AI-based multilingual medical examination summarization and translation service may generate summarization data 409 of medical examination contents by using a preset algorithm based on answer data. In another embodiment, the system may derive translation data 410 that are obtained by translating the summarization data 409 from a first language to a second language different from the first language by using a preset algorithm, based on the summarization data 409. In still another embodiment, the system may derive analysis data 411, including prediction disease information and a value of the chance of developing a prediction disease, by using a preset algorithm based on answer data.

When generating the summarization data 409, the system that provides the AI-based multilingual medical examination summarization and translation service may generate the summarization data 409, that is, a sentence, by using a neural network in which answer data having a form of term is used as input data.

Furthermore, the system that provides the AI-based multilingual medical examination summarization and translation service may recommend a hospital and a pharmacy (413) by using a preset algorithm based on the analysis data 411. In this case, the system may obtain location data of a user from the user, and may provide location data of a hospital and a pharmacy near the location of the user based on the obtained location data (413).

The present disclosure is not limited to the order illustrated in FIG. 4. Particularly, the order of the symptom start time 402 and additional measuring information 403 may be different from that illustrated in the drawings.

Operations of the server and the user terminal that are included in the system that provides the AI-based multilingual medical examination summarization and translation service, which generates summarization data based on answer data of a user, may be described as follows.

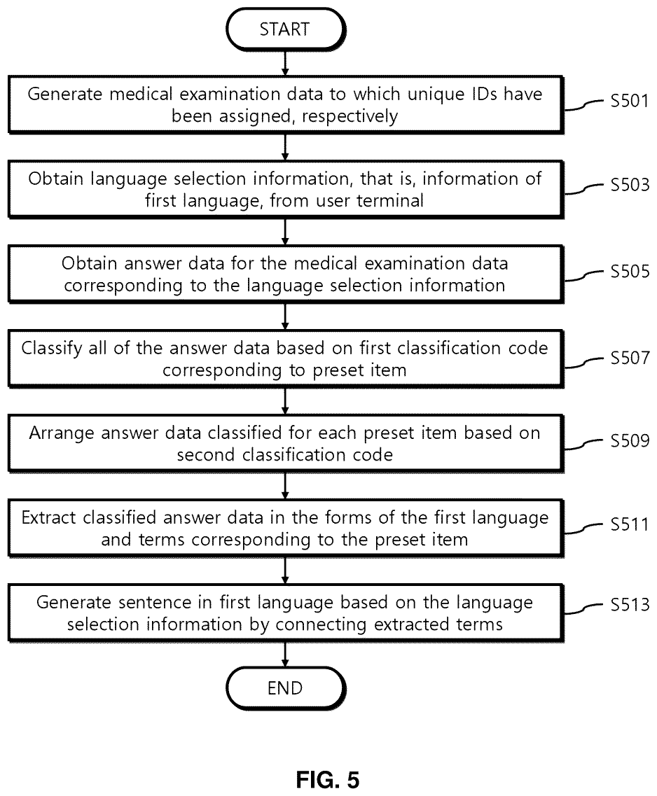

FIG. 5 is a flowchart illustrating an embodiment of a method of providing the AI-based multilingual medical examination summarization and translation service. The method of providing the AI-based multilingual medical examination summarization and translation service may be performed by the server of the system that provides the AI-based multilingual medical examination summarization and translation service.

Referring to FIG. 5, in step S501, the server may generate medical examination data to which unique IDs have been assigned, respectively. The medical examination data is a user's personal information and symptoms and/or disease-related data of the user, and include question information for a user's personal information and question information for symptoms and/or a disease of the user. The user's personal information may include height information, weight information, sex information, body temperature information, blood pressure information, pulse information, and respiration rate information of a user. Furthermore, the unique ID is an identifier for identifying and classifying medical examination data.

That is, the server may generate medical examination data, including question information and answer sheet information corresponding to each piece of question information. In this case, the server may assign a unique ID to each piece of question information and each piece of answer sheet information. Furthermore, the server previously stores, in the database, a plurality of linguistic expressions corresponding to one piece of symptom and/or disease information, and may make one unique ID correspond to a plurality of linguistic expressions that correspond to one piece of symptom and/or disease information. The plurality of linguistic expressions may include various forms of terms, such as a descriptive type or a word type for the same symptom or disease, various expression forms, such as a medical expression and an idiomatic expression for the same symptom or disease, and a term for each of the plurality of languages for the same symptom or disease.

For example, if one unique ID has been assigned to a symptom called stomachache, the unique ID corresponds to various forms of terms of stomachache, such as “stomachache”, “stomachache occurs”, and “by stomachache”. Furthermore, the unique ID of stomachache corresponds to “stomachache”, that is, a medical expression of stomachache, and a “tummyache”, “celialgia”, and “cardialgia”, that is, various idiomatic expressions of stomachache. Likewise, the unique ID of stomachache corresponds to “ [symbol] ” that is a language form of a Korean version, a “stomachache” that is a language form of an English version, and “ [symbol] ” that is a language form of a Chinese version. That is, such various expressions may be made to correspond to each other by one unique ID.

Furthermore, the unique ID may include first classification code and second classification code for classifying medical examination data. The first classification code is a code for primarily classifying medical examination data into a plurality of types. The second classification code is a code for secondarily classifying that medical examination data that have been primarily classified into a plurality of types corresponding to each of the first classification codes.

The first classification code may classify medical examination data as one of an independent data type, a dependent data type, a personal data type, and other data type. The independent data type is a symptom and/or disease-related data type having a form in which an additional description is required. The independent data type means a data type for a cardinal symptom and an accompanied symptom. Medical examination data may be classified into a cardinal symptom-related data type and an accompanied symptom-related data type even within the independent data type. For example, the independent data type may be a type in which symptom-related medical examination data, such as “stomachache”, “headache”, or “cough”, are classified. The dependent data type is a symptom and/or disease-related data type having a form in which the independent data type is described. The dependent data type means a data type for expressions that are dependent on a cardinal symptom and an accompanied symptom. Symptom start time-related information may also be classified as the dependent data type. For example, the dependent data type may be a type in which medical examination data that describe a symptom, such as a “squeezing pain” or “to the degree that a painkiller is required”, are classified.

The personal data type is a user's personal information-related data type. For example, the personal data type may include a vitals sign data type, a past history data type, a family history data type, and a social history data type. Finally, the other data type is a type that does not require an additional description and in which the independent data type is not described. The other data type means a data type that is not symptom and/or disease-related information, but is necessary upon medical treatment. For example, the other data type may be a type in which expressions describing the state of a user, such as a “tall and thin body” and “night sweat”, are classified.

The second classification code is a code for classifying medical examination data into data types for a plurality of pieces of aspect information related to types that have been classified by the first classification code, respectively. The plurality of pieces of aspect information may mean information that is additionally necessary in order to identify a symptom and/or a disease, among types classified by the first classification code. The aspect information may be additional information on a symptom and/or a disease that has been classified as the independent data type, among types classified by the first classification code. For example, the aspect information may include a progress condition (mitigate or deteriorate) related to a symptom and/or a disease, a symptom development location, size, and color, and a user's feeling of a symptom. That is, medical examination data may be primarily classified by the first classification code, and may be secondarily classified based on the second classification code, that is, a classification code for subsequent aspect information.

In step S503, the server may obtain language selection information, that is, information of a first language that is selected among preset languages, from the user terminal. In this case, the first language means the first language set by a user. The user may select one of a plurality of preset languages stored in the database. Thereafter, the server may provide medical examination data and summarization data that have been expressed in the first language selected by the user.

In step S505, the server may transmit, to the user terminal, medical examination data corresponding to the language selection information, and may obtain answer data for the medical examination data from the user terminal. That is, the server may transmit, to the user terminal, the medical examination data expressed in the first language. In this case, the answer data mean data, including medical examination data and answer information for the medical examination data. Furthermore, the answer data may include figure and/or photo data, in addition to text data expressed in a language.

In this case, the server may distinguish between first answer information that is selected by a user and second answer information that is not selected, within the medical examination data. Furthermore, the server may obtain, from the user terminal, answer data including the first answer information and/or second answer information for the medical examination data. The server may also obtain information on nonfeasance that is necessary in order to identify a symptom and/or a disease by obtaining the second answer information, that is, information that has not been selected, but the present disclosure is not limited thereto. The server may obtain only information corresponding to the first answer information, which is selected by the user within the medical examination data, as answer data from the user terminal.

The server may generate summarization data by classifying all of answer data based on a unique ID corresponding to a preset item and extracting linguistic expressions in the forms of the first language and the terms corresponding to the preset item.

Specifically, in step S507, the server may classify all of the answer data based on the first classification code corresponding to a preset item of summarization data. The server may set a unique ID corresponding to each item of summarization data. Accordingly, all of the answer data may be classified for each preset item within the summarization data by the first classification code.

In this case, the preset item within the summarization data may include “user's personal information, a cardinal symptom of a user, “the time when a disease developed”, “cardinal symptom and corresponding aspect information”, “one or more accompanied symptoms and corresponding aspect information”, “additional information”, “past history, family history, and social history information”.

Accordingly, answer data that are classified as the cardinal symptom-related data type that is included in the independent data type may be classified as “cardinal symptom of a user” and “cardinal symptom and corresponding aspect information” items within the summarization data. Furthermore, answer data that are classified as the accompanied symptom-related data type that is included in the independent data type may be classified as a “one or more accompanied symptoms and corresponding aspect information” item within the summarization data. Answer data that are classified as a data type to describe a cardinal symptom, which is included in the dependent data type, may be classified as the “cardinal symptom and corresponding aspect information” item within the summarization data. Likewise, answer data that are classified as the data type to describe an accompanied symptom, which is included in the dependent data type, may be classified as a “one or more accompanied symptoms and corresponding aspect information” item within the summarization data. Furthermore, answer data that are classified as a data type related to the symptom start time of a cardinal symptom, which is included in the dependent data type, may be classified a “time when a symptom developed” item within the summarization data.

The past history data type, the family history data type, and the social history data type that are included in the personal data type may be classified as “past history, family history, and social history information” items, respectively, within the summarization data. Finally, the other data type may be classified as an “additional information” item within the summarization data. Furthermore, information that is additionally input in the descriptive type by a user may be classified as an “additional information to be transmitted to a medical team” item within the summarization data.

In step S509, the server may classify and arrange the answer data classified for each preset item based on the second classification code. That is, all of the answer data may be classified for each preset item within the summarization data by the first classification code, and may be secondarily classified even within the preset item by the second classification code. Furthermore, all of the answer data may be arranged within the preset item by the second classification code.

That is, the server may classify the classified answer data as the “cardinal symptom and corresponding aspect information” item within the summarization data in a data type for cardinal symptom-related aspect information. Furthermore, the server may classify the cardinal symptom-related aspect information as a “corresponding aspect information” part even within the “cardinal symptom and corresponding aspect information” item. Furthermore, the server may arrange the answer data for a cardinal symptom in a front part of the “cardinal symptom and corresponding aspect information” item based on the second classification code, and may arrange answer data for cardinal symptom-related aspect information in a rear part of the “cardinal symptom and corresponding aspect information” item.

In step S511, the server may extract the classified answer data in a first language corresponding to the language selection information and forms of terms corresponding to the preset item. The server may extract the answer data in the form of linguistic expressions in order to visually derive the summarization data. In this case, the server may extract the first language corresponding to the language selection information and terms corresponding to the preset item, among a plurality of pre-stored linguistic expressions corresponding to the unique ID of the answer data.

For example, if the server has set a form of terms corresponding to a preset item in a descriptive form, the server may extract, from the database, a form of terms in a descriptive form term corresponding to a corresponding item, among a word form term and a descriptive form term that have been previously stored and that correspond to the unique ID of answer data. That is, if the server has set a preset item in the descriptive form, the server may extract the answer data in the form of a descriptive form term, such as “complain of stomachache” or “experience diarrhea symptoms”. In contrast, if the server has set a preset item in a word form, the server may extract the answer data in the form of a word form term, such as “stomachache” or “diarrhea”.

Furthermore, if the server has set the first language corresponding to the language selection information as English, the server may extract, from the database, linguistic expressions in English, that is, the first language corresponding to the language selection information, among a plurality of languages (e.g., Korean, Chinese, Japanese, and English) that have been previously stored and that correspond to the unique ID of answer data. That is, the server may extract the answer data in English “stomachache” or “diarrhea”.

In step S513, the server may generate a sentence in the first language based on the language selection information by connecting the extracted terms. That is, the server may generate summarization data including the sentence that is generated by connecting the terms of the extracted linguistic expressions. In this case, the server may generate the summarization data that have been expressed in the first language.

Furthermore, the server may perform an artificial intelligence (AI) training step for generating a sentence. Specifically, the server may train AI by using all terms extracted in the first language for each preset item as input data and using a sentence that has been completed in the first language as output data. AI may connect the terms through the AI training step, may determine a subject and predicate, and may output a sentence that does not have a problem in grammar of the first language.

In another embodiment, the server may communicate with an AI server including a large-scale language model. Accordingly, the server may input all terms extracted for each preset item into the AI server. The AI server may output and transmit, to the server, a sentence that is obtained by connecting all of the terms.

As the server outputs or receives all of the sentences for each preset item, summarization data can be generated.

In an embodiment, the server generates summarization data by classifying and arranging answer data for each of items within the summarization data by using a unique ID. Accordingly, efficiency and productivity of medical treatment can be improved because the time that is taken for medical examination and recording and writing, which occupy most of the medical treatment time, is reduced. Furthermore, medical records can be standardized because the server has a preset structure for each of items within summarization data based on unique IDs, and the amount of records can be increased due to efficiency of medical records.

The server summarizes and organizes medical examination through the system according to an embodiment of the present disclosure. Accordingly, the understanding of a medical team for the state of a patient can be increased because the medical team can previously determine the state of the patient based on summarization data. Furthermore, the quality of a medical service that is provided to a patient can be improved because the patient can be matched with a proper medical department.

Furthermore, a multilingual version of summarization data can be derived because the server generates summarization data by extracting linguistic expressions corresponding to each item within the summarization data based on a unique ID through the system according to an embodiment of the present disclosure. Accordingly, a user (or patient) can be easily provided with a medical service although the user is in a place where there is a language barrier because verbal communication between the user and a medical team is easily performed. Furthermore, misdiagnosis by a medical team and the misuse and overuse of medical drugs can be minimized because information that is missed due to a language barrier between the user and the medical team can be minimized.

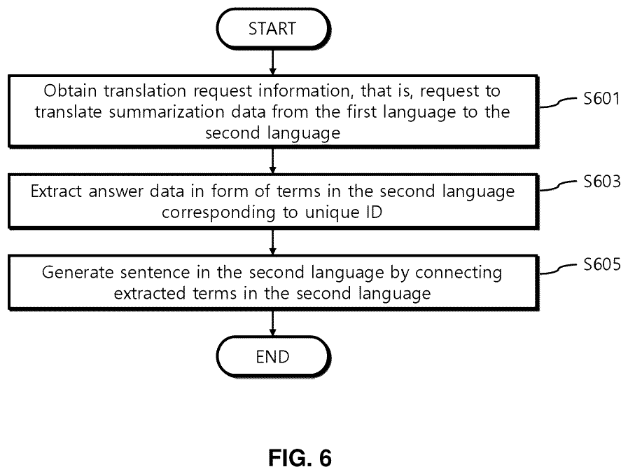

FIG. 6 is a flowchart illustrating an embodiment of a method of providing a translation service by the system that provides the AI-based multilingual medical examination summarization and translation service. The method of providing a translation service may be performed by the server of the system that provides the AI-based multilingual medical examination summarization and translation service.

Referring to FIG. 6, in step S601, the server may obtain translation request information, that is, a request to translate summarization data from a first language to a second language, from the user terminal. That is, if a user wants to receive the summarization data expressed in the second language different from the first language, the user terminal may transmit the translation request information to the server.

First, the server may obtain language selection information for the first language from the user terminal, may provide the user terminal with medical examination data expressed in the first language, and may generate summarization data expressed in the first language.

After the server generates the summarization data expressed in the first language, the server may obtain translation request information, that is, a request to translate the summarization data from the first language to the second language, from the user terminal.

In step S603, the server may extract answer data in the form of terms in the second language, corresponding to the unique ID of the answer data, based on the translation request information. That is, the server may extract the answer data for each of items within the summarization data in the form of linguistic expressions in the second language, instead of the first language, based on the translation request information.

In step S605, the server may generate a sentence in the second language by connecting the extracted terms in the second language. The server may generate the sentence through AI in the same was as the server generates the sentence in the first language.

In this case, the server may perform an AI training step. The server may train AI by using all terms extracted in one language, among multiple languages that have been previously stored for each preset item, as input data and using a sentence expressed in the corresponding language as output data. That is, the server may train AI for each language.

In another embodiment, the server may communicate with an AI server including a large-scale language model. Accordingly, the server may input all terms extracted in one language, among multiple languages that have been previously stored for each preset item, into the AI server. The AI server may output and transmit, to the server, the sentence expressed in the corresponding language by connecting all of the terms.

As the server outputs or receives all sentences that have been expressed in the second language for each preset item, translation data that have been expressed in the second language can be generated.

A detailed step of obtaining the answer data for the medical examination data from the user terminal and a step of providing diagnosis information for the answer data may be described as follows.

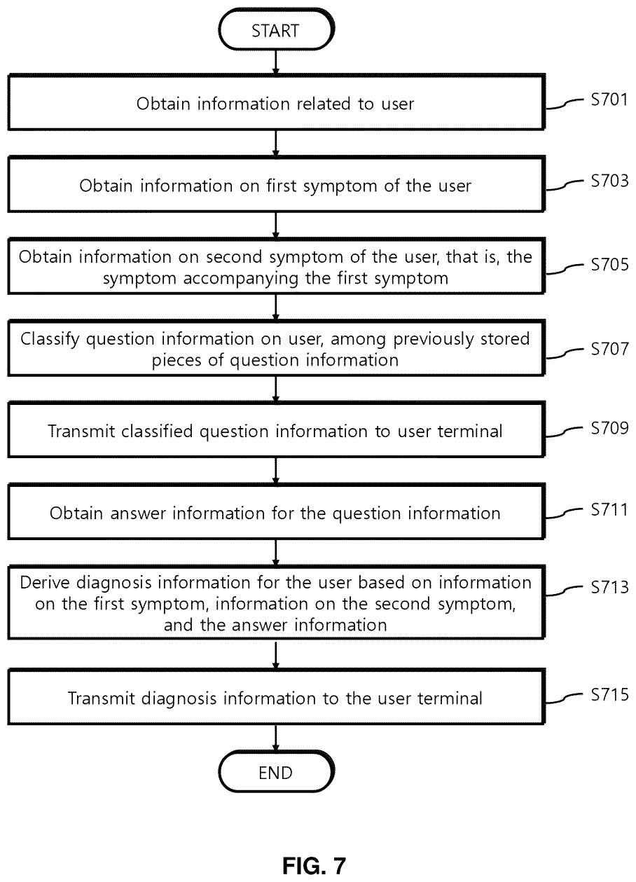

FIG. 7 is a flowchart illustrating an embodiment of a method of providing diagnosis information by the system that provides the AI-based multilingual medical examination summarization and translation service. The method of providing diagnosis information may be performed by the server of the system that provides the AI-based multilingual medical examination summarization and translation service.

The server may obtain answer data for medical examination data from the user terminal.

Specifically, in step S701, the server may obtain information related to a user from the user terminal. In this case, the information related to a user may include user's personal information, information on the social history of the user, and vitals sign information of the user. Body measuring information of the user may include information on the height and weight of the user. Furthermore, the information on the social history of the user may include information on a smoking state, smoking frequency, smoking duration, a drinking state, drinking frequency, and drinking duration of the user.

In this case, the vitals sign information may include numerical value information, such as body temperature of the user, blood pressure of the user, a heart rate of the user, a respiration rate of the user, oxygen saturation of the user, and blood glucose of the user. In an embodiment, the server may obtain bio information of the user based on values input by the user, from the user terminal. According to another embodiment, the server may obtain, from the user terminal, bio information of the user based on values measured by a device that is connected to the user terminal.

In step S703, the server may obtain information on a first symptom of the user from the user terminal. In this case, the first symptom may mean one major symptom, among one or more symptoms that the user is now experiencing. In this case, the information on the first symptom may include information on the name of the first symptom and the time when the first symptom developed. The server may determine whether the first symptom is acute/chronic and obtain information on whether the first symptom is acute/chronic, based on the information on the time when the first symptom developed.

In an embodiment, when a user inputs a linguistic expression related to a symptom through the user terminal, the server may classify information on symptoms corresponding to the linguistic expression that is obtained from the user terminal, among pieces of information on symptoms stored in the database. Furthermore, the server may transmit, to the user terminal, the information on the symptoms corresponding to the linguistic expression that is obtained from the user terminal. Accordingly, the user can input accurate information on the symptom although the user inputs an idiomatic linguistic expression.

In an embodiment, the server may obtain history information related to a disease and/or an illness from the user terminal. In this case, the history information may include information on the past history of a disease and/or an illness that a user has experienced from the past, the family history of a disease and/or an illness that a family member of a user experienced or is experiencing, and drugs that a user has recently taken or has a history of taking.

Furthermore, the server may preferentially transmit, to the user terminal, information on the past history, family history, and history of taking drugs, which have relevance with the first symptom, among pieces of information on past histories, family histories, and histories of taking drugs stored in the database. In this case, the relevance may be determined based on information on medical departments related to a symptom, the past history, the family history, and the history of taking drugs, respectively.

In step S705, the server may obtain information on a second symptom from the user terminal. In this case, the information on the second symptom may refer to a symptom that accompanies the first symptom, among one or more symptoms that the user is now experiencing. In this case, the information on the second symptom may include information, such as the name of the second symptom and the time when the second symptom developed. In this case, the second symptom may include a plurality of symptoms.

In an embodiment, the server may transmit, to the user terminal, information on symptoms that commonly develop in patients by grouping the information on the symptoms, and may transmit, to the user terminal, information on symptoms that have been classified on the basis of medical departments by grouping the information on the symptoms. Furthermore, the server may preferentially transmit information on symptoms having relevance with the first symptom, among pieces of information on symptoms stored in the database, to the user terminal. In this case, the relevance may be determined based on information on a medical department that is related to each of the symptoms.

In an embodiment, when a user inputs a linguistic expression related to a symptom through the user terminal, the server may classify information on symptoms corresponding to the linguistic expression that is obtained from the user terminal, among pieces of information on symptoms stored in the database. Furthermore, the server may transmit, to the user terminal, the information on the symptoms corresponding to the linguistic expression that is obtained from the user terminal. Accordingly, the user can input accurate information on the symptom although the user inputs an idiomatic linguistic expression.

In step S707, the server may classify question information regarding the user, among pieces of question information that have been previously stored. Specifically, the server may classify question information regarding the user, among pieces of question information that have been previously stored in the database, based on information that is obtained from the user terminal. In this case, the information that is obtained from the user terminal may include information related to the user, information on the first symptom of the user, information a history of a disease and/or illness of the user, and information on the second symptom of the user.

The database may record information on one or more questions. The one or more questions may include a question about symptoms of a user and a question about a disease of a user. In this case, the one or more questions may have a corresponding relation with the symptoms of the user and/or the disease of the user.

The one or more questions may be questions having different formats. In an embodiment, the format of one or more questions may be one formation, among the formats of an either-or type question, a multiple-choice single selection question, a multiple-choice multiple-selection question, a question for selecting a developing portion of a symptom, and a question for selecting a criterion on which a degree of a symptom is digitized.

In step S709, the server may transmit the classified question information to the user terminal. In this case, the question information may include a question, reference data such as an image that is incidental on the question, and a selection sheet for the question.

In step S711, the server may obtain answer information for the question information from the user terminal.

In step S713, the server may derive diagnosis information for the user, based on the information on the first symptom, the information on the second symptom, and the answer information. In this case, the diagnosis information may include information on a medical department that is derived based on information obtained from the user, at least one disease having a good possibility that the disease developed in the user, and information on the emergency of a disease.

In step S715, the server may transmit the diagnosis information to the user terminal.

That is, the server may derive the diagnosis information for the user by comprehensively considering the answer information for the question related to the user, in addition to the information related to the user, which is obtained from the user terminal, the information on the first symptom of the user, the information on the history of a disease and/or illness of the user, and the information on the second symptom of the user. In this case, the question related to the user includes a question about a symptom and a question about a disease. The answer information for the question related to the user may also include answer information related to a symptom and answer information related to a disease.

In an embodiment, the server may preferentially classify a question about a symptom based on user-related information and transmit the question about the symptom to the user terminal. Furthermore, when receiving answer information for the question about the symptom, the server may classify a question about a disease by additionally incorporating the answer information for the question about the symptom in addition to the user-related information, and may transmit the question about the disease to the user terminal.

Accordingly, the server can improve the precision of derived diagnosis results by comprehensively analyzing user-related information, answer information for a question about a symptom, and answer information for a question about a disease.

Operations for obtaining information for the operations that are illustrated in FIGS. 5 to 7 and that are performed by the system that provides the AI-based multilingual medical examination summarization and translation service may be performed in interface described hereinafter.

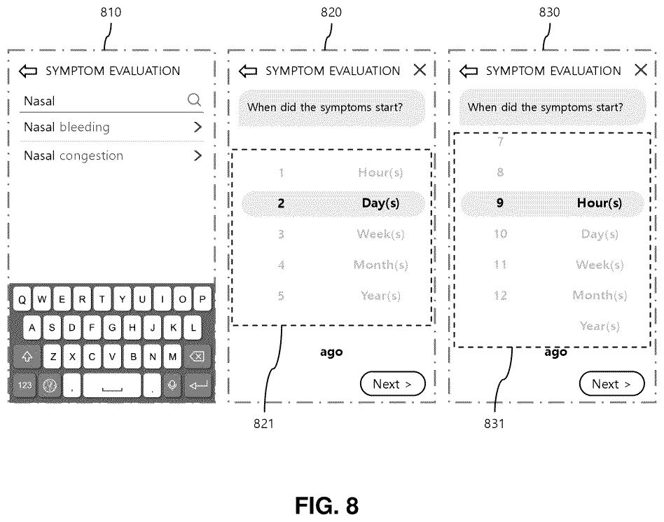

FIG. 8 is a diagram illustrating an embodiment of interfaces for obtaining information on a first symptom of a user, which is output to a user terminal.

Referring to FIG. 8, the interface for obtaining information on the first symptom of a user may include an interface 810 for inputting the name of the first symptom and interfaces 820 and 830 for inputting the time when the first symptom developed.

The interface 810 for inputting the name of the first symptom may include an area 811 for symptom search and an area 813 in which an associated word is displayed based on terms input to the area 811 for symptom search.

A user may input a term for expressing the first symptom to the area 811 for symptom search. The system that provides the AI-based multilingual medical examination summarization and translation service may provide information on symptoms corresponding to the term that is input by the user. Accordingly, symptom information and/or associated information corresponding to the term that is input by the user may be displayed in the interface 810 for inputting the name of the first symptom.

The user may select a button corresponding to the first symptom, among buttons corresponding to words displayed in the area 813 in which associated words are displayed. Accordingly, the system may obtain answer data for medical examination data related to the first symptom.

The interfaces 820 and 830 for inputting the time when the first symptom developed may include areas each for inputting and outputting number information for expressing the time when the first symptom developed and areas 821 and 831 each for inputting and outputting information for expressing a time unit (e.g., hourly, daily, monthly, or yearly), respectively.

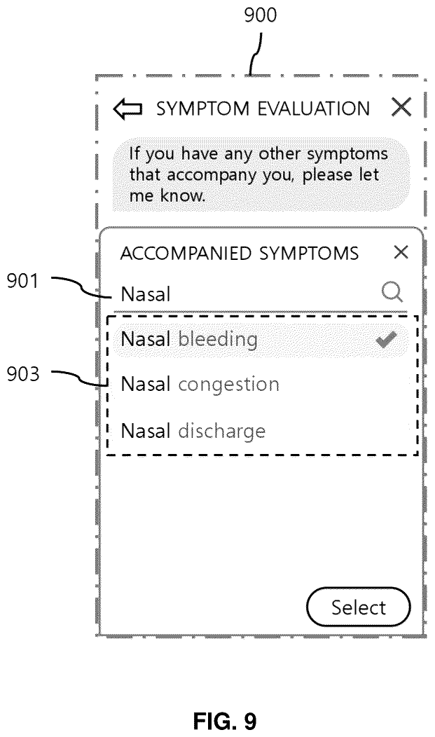

FIG. 9 is a diagram illustrating an embodiment of an interface for obtaining information on a second symptom.

Referring to FIG. 9, an interface 900 for inputting the name of the second symptom may include an area 901 for symptom search and an area 903 for outputting associated words based on terms that is input to the area 901 for symptom search.

A user may input a term for expressing the second symptom to the area 901 for symptom search. The system that provides the AI-based multilingual medical examination summarization and translation service may provide information on symptoms corresponding to the term that is input by the user. Accordingly, symptom information and/or associated information corresponding the term that is input by the user may be displayed in the interface 900 for inputting the name of the second symptom.

The user may select a button corresponding to the second symptom, among buttons corresponding to words displayed in the area 903 in which associated words are displayed. Accordingly, the system may obtain answer information for medical examination data related to the second symptom.

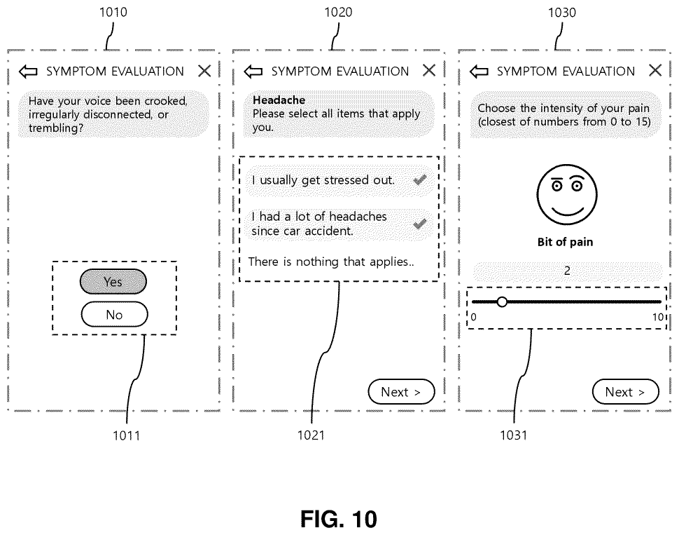

FIG. 10 is a diagram illustrating an embodiment of interfaces for displaying question information regarding a symptom and/or disease of a user, which is output to the user terminal.

Referring to FIG. 10, the interface for outputting medical examination data related to symptoms and/or a disease of a user may include an area for outputting a question and areas 1011, 1021, and 1031 each for outputting an answer sheet for a question and inputting answer information. The areas 1011, 1021, and 1031 for inputting answer information for a question may be differently output on the interfaces depending on the type of question.

The area 1011 for inputting answer information for a question in an interface 1010 for outputting either-or type question information may include buttons for selecting positive information (“Yes”) for a question and negative information (“No”) for the question.

The area 1021 for inputting answer information for a question in an interface 1020 for outputting multiple-choice single or multiple-selection type question information may include buttons for selecting information on a plurality of selection sheets for a question.

The area 1031 for inputting answer information for a question in an interface 1030 for obtaining information on the intensity of a pain attributable to a symptom may include a button that is movable depending on the intensities (0 to 10) of a pain.

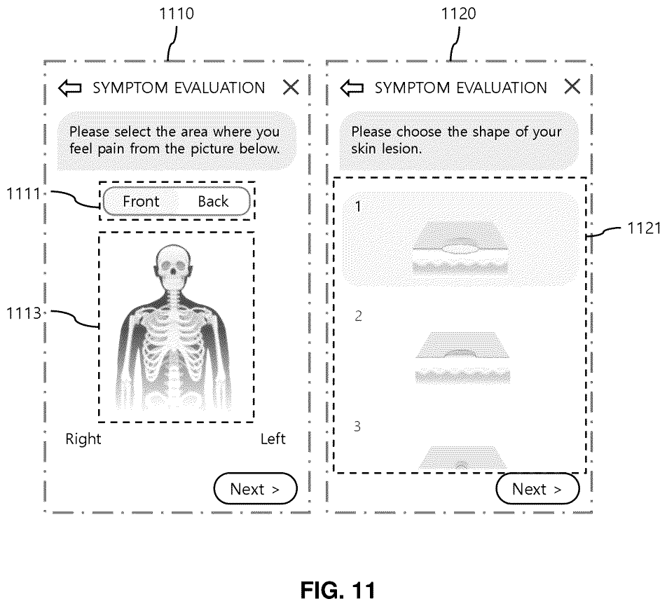

FIG. 11 is a diagram illustrating an embodiment of interfaces for displaying question information regarding a symptom and/or disease of a user, which is output to the user terminal.

Referring to FIG. 11, the interfaces for outputting medical examination data related to a symptom and/or disease of a user may include an area for outputting a question and areas 1111, 1113, and 1121 for outputting an answer sheet for a question and inputting answer information. The areas 1111, 1113, and 1121 for inputting answer information for a question may be differently output in the interfaces depending on the type of question.

An interface 1110 for obtaining information on a symptom development location may include the area 1111 for inputting answer information related to the front and rear of a human body and the area 1113 for outputting an image object of a human body and inputting answer information. A user may input a signal to a body portion corresponding to a symptom developing portion in the area 1113 for outputting an image object of a human body.

Furthermore, an interface 1120 for obtaining information on a symptom development shape may include an area 1121 including buttons for selecting information on a plurality of selection sheets for a question.

In this case, when an image corresponding to question information and/or answer information is present, an image corresponding to the question information and/or answer information may be displayed in the interface for outputting question information.

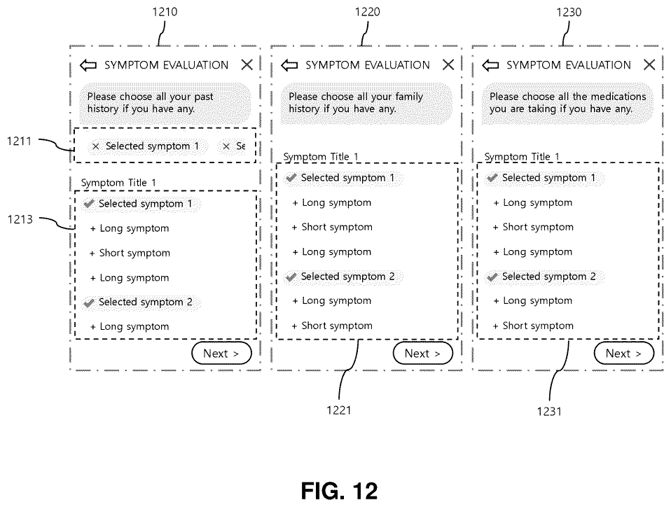

FIG. 12 is a diagram illustrating an embodiment of interfaces for obtaining information on the symptom and/or disease-related history of a user, which is output to the user terminal.

Referring to FIG. 12, interfaces 1210, 1220, and 1230 for inputting information on the symptom and/or disease-related history of a user may include an area 1213 in which at least one object related to the past history of a user is displayed in the form of a button, an area 1221 in which at least one object related to the family history of a user is displayed in the form of a button, and an area 1231 in which at least one object related to a user's history of taking drugs is displayed in the form of a button, respectively.

Furthermore, the interfaces 1210, 1220, and 1230 for inputting information on the symptom and/or disease-related history of a user may include an area 1211 for outputting at least one inputted object.

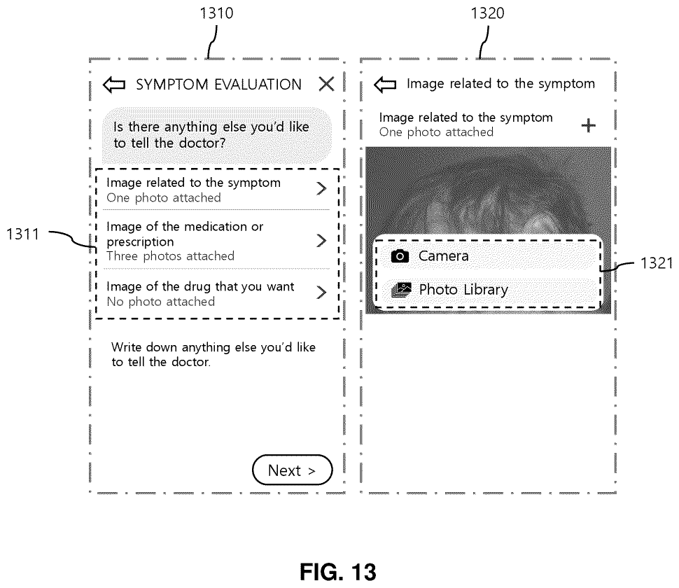

FIG. 13 is a diagram illustrating an embodiment of interfaces for obtaining symptom and/or disease-related additional information of a user, which is output to the user terminal.

Referring to FIG. 13, an interface 1310 for obtaining additional information related to a symptom and/or disease of a user may include an area 1311 for inputting an image related to a symptom, drugs being taken or a prescription image for the drugs, and an image of drugs that a patient want to prescribe.

Furthermore, an interface 1320 for obtaining additional information related to a symptom and/or disease of a user based on input information of the user may include an area 1321 including a button for selecting an image captured by the user terminal and a button for moving to a camera function of the user terminal.

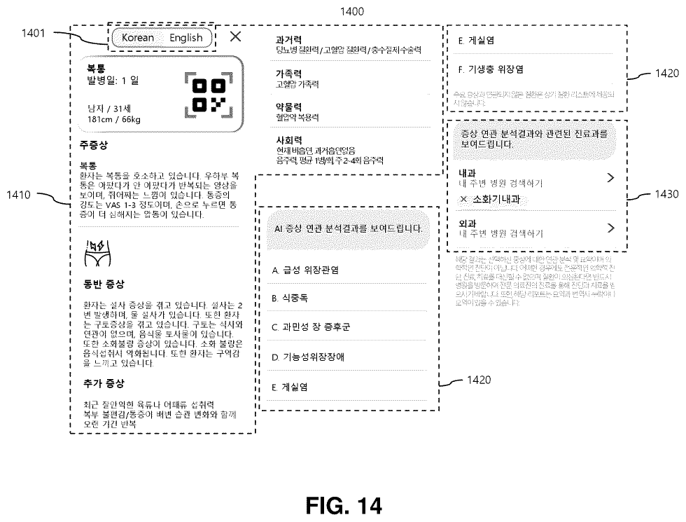

FIG. 14 is a diagram illustrating an embodiment of interfaces for displaying summarization information and diagnosis information of a user, which are output to the user terminal.

Referring to FIG. 14, an interface 1400 for outputting report data including summarization information and diagnosis information of a user may include an interface 1410 for outputting summarization information of a user, an interface 1420 for outputting diagnosis information of a user, and an interface 1430 for outputting recommended hospital and/or pharmacy information, which are provided by the system that provides the AI-based multilingual medical examination summarization and translation service. Furthermore, the interface 1400 for outputting report data may further include an area 1401 in which language information that expresses summarization information and diagnosis information is displayed.

Information on a cardinal symptom (or a first symptom) of a user, information on an accompanied symptom (or a second symptom) of a user, additional input information of a user, information on the past history of a user, information on the family history of a user, information on a user's history of taking drugs, and information on the social history of a user may be displayed in the interface 1410 for outputting summarization information of a user.

Information on a predicted disease of a user based on diagnosis information of the user may be displayed in the interface 1420 for outputting diagnosis information.

Information on a medical department that is now suitable for a user based on obtained information on the location of the user and location information related to a hospital adjacent to a user based on location data may be displayed in the interface 1430 for outputting recommended hospital and/or pharmacy information.

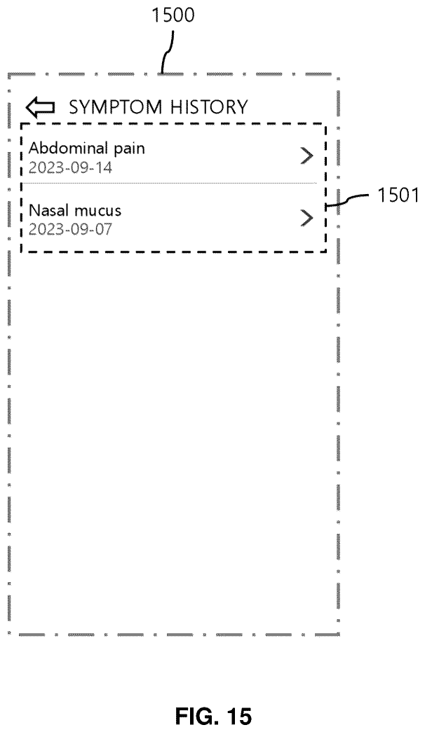

FIG. 15 is a diagram illustrating an embodiment of an interface in which summarization information past histories are expressed in Korean in order to output the summarization information past history of a user, which is output to the user terminal.

Referring to FIG. 15, an interface 1500 for outputting the summarization information past history of a user may include an area 1501 for outputting the past symptom and/or disease-related history of a user. The user terminal may output the summarization information past history of a user again based on input information of the user.

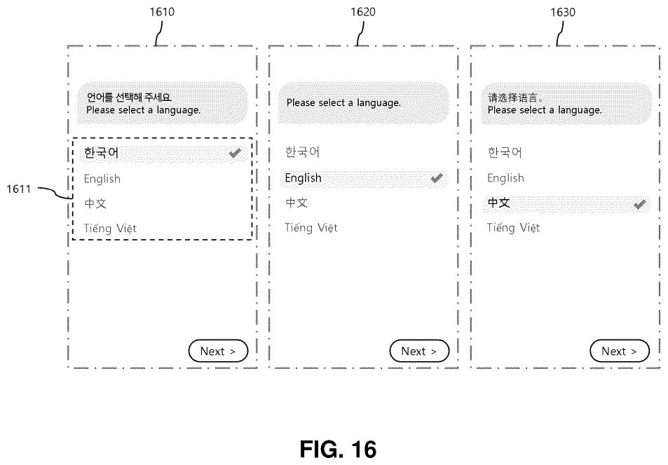

FIG. 16 is a diagram illustrating an embodiment of interfaces for obtaining language selection information of a user, which are output to the user terminal.

Referring to FIG. 16, an interface 1610 for obtaining language selection information of a user may include an area 1611 including buttons for selecting a plurality of preset languages. A user may input information on one language by selecting one of the buttons for selecting the plurality of preset languages in the area 1611. A language in a subsequent interface may be set based on language selection information, that is, input information of a user.

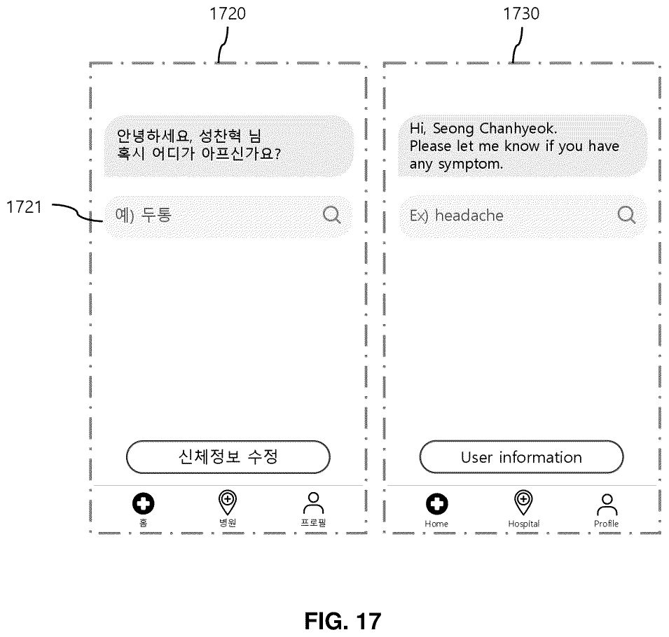

FIG. 17 is a diagram illustrating an embodiment of interfaces in which different languages are expressed in order to obtain information on a first symptom of a user, which is output to the user terminal.

Referring to FIG. 17, an interface for obtaining information on the first symptom of a user may include an interface 1720 expressed in Korean and an interface 1730 is expressed in English. The interfaces 1720 and 1730 for inputting the name of the first symptom may include an area 1721 for symptom search.

The interface for obtaining information on the first symptom of a user may be expressed in Korean only or expressed in English only based on language selection information of a user. However, Korean and English are exemplary, and an embodiment of the present disclosure is not limited thereto.

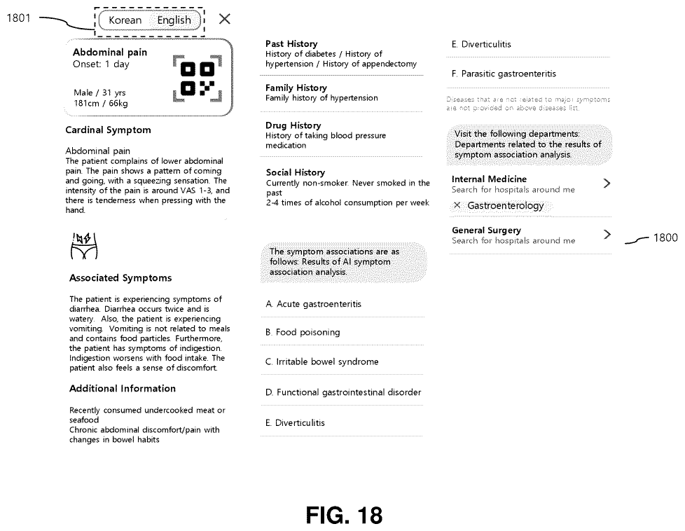

FIG. 18 is a diagram illustrating an embodiment of an interface for outputting summarization information and diagnosis information of a user to the user terminal in English.

Referring to FIG. 18, an interface 1800 for outputting report data including summarization information and diagnosis information of a user in English is an interface for outputting the interface 1400 for outputting, in English, report data including summarization information and diagnosis information of a user, which is output in Korean in FIG. 14. Accordingly, the interface 1800 for outputting report data including summarization information and diagnosis information of a user in English may include an interface for outputting summarization information of a user, an interface for outputting diagnosis information of a user, an interface for outputting recommended hospital and/or pharmacy information, and an area 1801 in which language information used in the report data is selected, which are provided by the system.

In an embodiment, the interface 1800 for outputting report data in English may be an interface in which language selection information of a user is displayed in English. In another embodiment, the interface 1800 for outputting report data in English may be an interface in which input information is received in English through the area 1800 in which language information is selected and a preset another language is translated and displayed in English.

The methods according to embodiments of the present disclosure may be implemented in the form of program instructions which may be performed through various computer means and recorded on a computer-readable medium. The computer-readable medium may include a program instruction, a data file, and a data structure solely or in combination. The program instruction that is recorded on the computer-readable medium may be specially designed and constructed for the present disclosure or may be known and available to those skilled in computer software.

Examples of the computer-readable medium include hardware devices that are specially constructed to store and execute program instructions, such as ROM, RAM, and flash memory. Examples of the program instruction include a high language code which may be executed by a computer by using an interpreter, in addition to a machine code, such as that produced by a compiler. The hardware device may be constructed to operate as at least one software module in order to perform an operation of the present disclosure, and vice versa.

Although the present disclosure has been described with reference to the embodiments, those skilled in the art may understand that the present disclosure may be modified and changed in various ways without departing from the spirit and scope of the present disclosure written in the claims.

What is claimed is:

1. An artificial intelligence (AI)-based multilingual medical examination summarization method being performed by a processor of a server, the method comprising:
generating medical examination data to which unique IDs have been assigned, respectively, wherein the medical examination data comprise user's personal information and symptom and/or disease-related data of the user;
obtaining language selection information that is information of a first language, selected among preset languages, from user terminal;
obtaining answer data for the medical examination data corresponding to the language selection information from the user terminal; and
classifying all of the answer data based on a unique ID corresponding to a preset item and generating summarization data by extracting a linguistic expression in the forms of the first language and terms corresponding to the preset item.

2. The method of claim 1, wherein the unique ID comprises:
a first classification code to primarily classify the medical examination data into a plurality of types; and
a second classification code to secondarily classify the primarily classified medical examination data into a plurality of types corresponding to each of the first classification codes.

3. The method of claim 2, wherein the generating of the summarization data comprises:
classifying all of the answer data based on the first classification code corresponding to the preset item;
classifying and arranging the answer data that have been classified for each preset item based on the second classification code;
extracting the classified answer data in the forms of the first language corresponding to the language selection information and the terms corresponding to the preset item; and
generating a sentence in the first language based on the language selection information by connecting the extracted terms.

4. The method of claim 3, wherein the extracting of the classified answer data in the forms of the first language corresponding to the language selection information and the terms corresponding to the preset item comprises extracting the answer data in the first language corresponding to the language selection information and the form of the term corresponding to the preset item, among a plurality of pre-stored linguistic expressions corresponding to the unique ID of the answer data.

5. The method of claim 3, wherein:
the generating of the sentence in the first language based on the language selection information by connecting the extracted terms comprises AI training, and
the AI training comprises performing AI training by using all terms extracted for each preset item as input data and using a sentence completed in the first language as output data.

6. The method of claim 2, wherein the first classification code is a code to classify the medical examination data as one of an independent data type that is a type in which an additional description is required, a dependent data type that is a type in which the independent data type is described, a personal data type for the user's personal information, and other data type that is a type that does not require an additional description and in which the independent data type is not described.

7. The method of claim 6, wherein the second classification code is a code to classify the primarily classified medical examination data into a data type for a plurality of pieces of aspect information related to the types classified by the first classification code, respectively.

8. The method of claim 1, further comprising translating the summarization data,
wherein the translating of the summarization data comprises:
obtaining, from the user terminal, translation request information that is a request to translate the summarization data from the first language to a second language except the first language, among the preset languages;
extracting the answer data in a form of terms of the second language corresponding to the unique ID of the answer data based on the translation request information; and
generating a sentence in the second language by connecting the extracted terms in the second language.

9. The method of claim 1, wherein the obtaining of the answer data comprises:
distinguishing between first answer information that is selected by the user and second answer information that is not selected, within the medical examination data; and
obtaining, from the user terminal, answer data comprising the first answer information and/or the second answer information for the medical examination data.

10. The method of claim 1, wherein the obtaining of the answer data comprises:
obtaining information on a first symptom of the user from the user terminal;
obtaining, from the user terminal, information on a second symptom of the user that is a symptom accompanying the first symptom;
classifying question information regarding the user, among pieces of pre-stored question information based on the information obtained from the user terminal;
transmitting the classified question information to the user terminal; and
obtaining answer information for the question information from the user terminal.

11. A computer program stored in a computer-readable storage medium, wherein when the computer program is executed by a processor of an apparatus, an artificial intelligence (AI)-based multilingual medical examination summarization method is performed by the processor of the apparatus and comprises:
generating medical examination data to which unique IDs have been assigned, respectively, wherein the medical examination data comprise user's personal information and symptom and/or disease-related data of the user;
obtaining language selection information that is information of a first language, selected among preset languages, from a user terminal;
obtaining, from the user terminal, answer data for medical examination data corresponding to the language selection information; and
classifying all of the answer data based on a unique ID corresponding to a preset item and generating summarization data by extracting a linguistic expression in the forms of the first language and terms corresponding to the preset item.

12. The computer program of claim 11, wherein the unique ID comprises:
a first classification code to primarily classify the medical examination data into a plurality of types; and
a second classification code to secondarily classify the primarily classified medical examination data into a plurality of types corresponding to each of the first classification codes.

13. The computer program of claim 12, wherein the generating of the summarization data comprises:
classifying all of the answer data based on the first classification code corresponding to the preset item;
classifying and arranging the answer data that have been classified for each preset item based on the second classification code;
extracting the classified answer data in the forms of the first language corresponding to the language selection information and the terms corresponding to the preset item; and
generating a sentence in the first language based on the language selection information by connecting the extracted terms.

14. An artificial intelligence (AI)-based multilingual medical examination summarization apparatus comprising:
a storage unit in which at least one program instruction is stored; and
a processor configured to perform the at least one program instruction,
wherein the processor
generates medical examination data to which unique IDs have been assigned, respectively, wherein the medical examination data comprise user's personal information and symptom and/or disease-related data of the user;
obtains language selection information that is information of a first language, selected among preset languages, from a user terminal;
obtains answer data for the medical examination data corresponding to the language selection information from the user terminal; and
classifies all of the answer data based on a unique ID corresponding to a preset item and generates summarization data by extracting a linguistic expression in the forms of the first language and terms corresponding to the preset item.

15. The apparatus of claim 14, wherein the unique ID comprises:
a first classification code to primarily classify the medical examination data into a plurality of types; and
a second classification code to secondarily classify the primarily classified medical examination data into a plurality of types corresponding to each of the first classification codes.

16. The apparatus of claim 15, wherein in generating the summarization data, the processor
classifies all of the answer data based on the first classification code corresponding to the preset item,
classifies and arranges the answer data that have been classified for each preset item based on the second classification code;
extracts the classified answer data in the forms of (the first language corresponding to the language selection information and the terms corresponding to the preset item, and
generates a sentence in the first language based on the language selection information by connecting the extracted terms.
